(19) United States
(12) Patent Application Publication
(10) Pub. No.: US 2002/0091369 A1
(43) Pub. Date: Jul. 11, 2002

(54) Absorbent article

(76) Inventor: Roy Hansson, Alegardsgatan (SE)

Correspondence Address: YOUNG & THOMPSON, 745 SOUTH 23RD STREET 2ND FLOOR, ARLINGTON, VA, 22202

(21) Appl. No.: 10/022,373

(22) Filed: Dec. 20, 2001

Related U.S. Application Data
(60) Provisional application No. 60/256,482, filed on Dec. 20, 2000.

(30) Foreign Application Priority Data
Dec. 20, 2000 (SE) 0004759-7

(51) Int. Cl. A61F 13/15; A61F 13/20

(52) U.S. Cl. 604/392; 604/385.01

(57) ABSTRACT

Absorbent article such as a diaper and an incontinence guard comprising a pair of belt portions (9, 10) attached to the rear portion (6) of the article and which are intended to be fastened together around the waist of the wearer, wherein the one belt portion (9, 10) carries first fastening means (11) intended to be attached against the opposite belt portion (9,10) and where said front portion (5) exhibits second fastening means (8) intended to be attached to the belt portions (9, 10), in such a way that the article will assume a pantlike shape, where the belt portions (9, 10) form a part of the waist portions of the pant. Each belt portions (9, 10) are provided with at least one indicium (12), being placed on a appropriate distance from the attachment (13) of each belt portion (9, 10) to the rear portion (6) of the article, whereby each indicium (12) on one belt portion (9) has its corresponding indicium on the on the opposite belt portion (10).

### TECHNICAL FIELD

The present invention relates to an absorbent article such as a diaper and an incontinence guard comprising a liquid permeable topsheet, a liquid impermeable backsheet and an absorbent body enclosed therebetween, wherein the article having a front portion, a rear portion and, a crotch portion therebetween, and her is provided with a pair of belt portions attached to the rear portion of the article and which are intended to be fastened together around the waist of the wearer and wherein one belt portion is provided with first fastening means intended to be attached against the other belt portion and where said front portion is provided with attachment means intended to be attached to the belt portions, in such a way that the article will assume a pantlike shape, where the belt portions form a pt of the waist portions of the pant.

### BACKGROUND OF THE INVENTION

Diapers and incontinence guards usually exhibit a garment portion holding an absorbent body in place against the user's body and attachment means, which hold the garment portion in place also when the user is moving. A common type of attachment means are adhesive tapes or hook and loop fasteners of the touch-and-close type, which directly attach the front and rear portions of the absorbent article to each other. It is further known, through e.g., EP-A-0 287 388, BP-A-0 409 307, EP-A-0 528 282, EP-A-0 605 012 and FRA-2 586 558, to attach the front and rear portions of the article by means of a belt, wherein the possibilities to adjust the fit are improved. The belt also lead to a simplified change of the diaper and incontinence guard, respectively, in particular on a standing person.

A conventional diaper for children is in general applied having the child in a lying position. The attachment means are usually arranged on the rear portion and are attached to the front portion. This kind of application often requires the aid from another person. However, for an adult user it is more desirable to self be able to apply the incontinence guard. On a common type of belt diaper, the belt portions are therefore first attached around the waist. When the incontinence guard is fixed around the waist in this way, the user may reach after the rest of the incontinence guard between the legs and then the crotch portion of the incontinence guard is applied in the correct position by fastening the front portion of the diaper to the outside of te belt portions using hook and loop fasteners or tape tabs being arranged on the front portion and/or the belt portion. This design makes also possible for nursing personnel to apply the diaper on a standing person or for the user to apply the diaper on himself/herself in a standing position.

One problem for the user or the nursing personnel is to know whether the absorbing diaper pa of the article is centred oh the back of the user. When the belt is applied, i.e., when the belt portions is connected to each other on the belly of the wearer, it is difficult to know in which way the diaper part of the article is fit on the user, since one can not visually see how the article fits on the back. When the front portion is attached against the belt portions, it can easily happen that the article, if it has ended up wrong around the wearer, not provides the guard it is intended to provide and maybe cause discomfort to the wearer.

Therefore there is a need for absorbent article provided with a belt, wherein the belt adapts itself to the user using the diaper and also feels comfortable to wear.

### SUMMARY OF THE INVENTION

The object of the present invention is to provide a belted diaper or incontinence guard being comfortable to wear and which fits persons having different sizes This object is being solved in that each belt portion is provided with at least one indicium, being placed on a appropriate distance from the attachment of each belt portion to the rear portion of the article, whereby each indicium on one belt portion has its corresponding indicium on the opposite belt portion.

### BRIEF DESCRIPTION OF DRAWINGS

The invention will in the following be closer described with reference to an embodiment shown in the accompanying drawing.

FIG. 1 schematically shows a perspective view of a diaper or incontinence guard according to the invention.

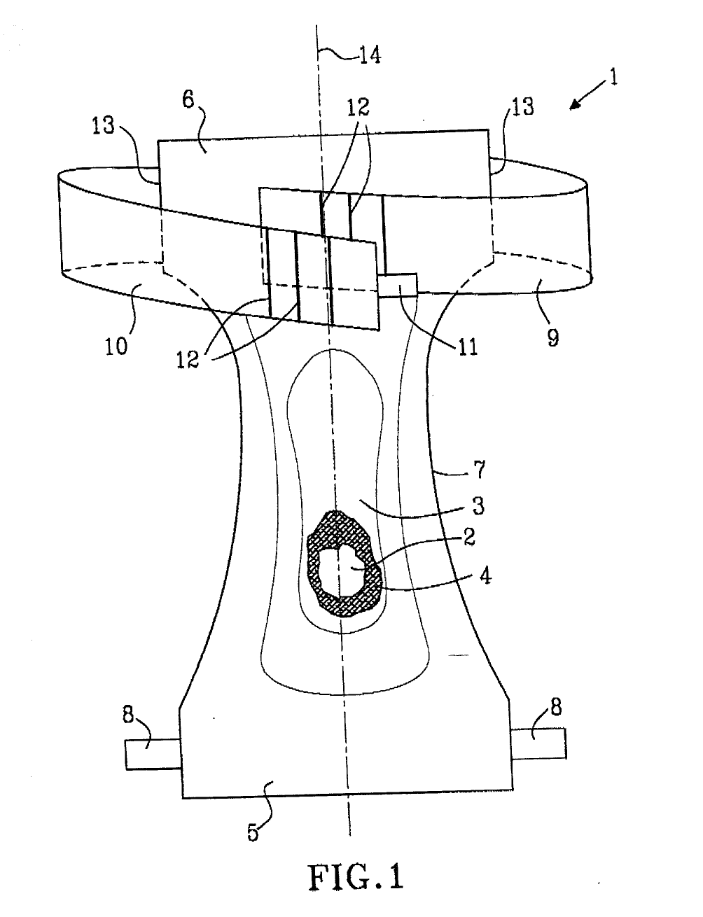

### DETAILED DESCRIPTION OF THE INVENTION

The drawing shows an embodiment of a diaper or incontinence guard 1 comprising a liquid impermeable backsheet 2, a liquid permeable topsheet 3 and at absorbent body 4 enclosed therebetween. The liquid permeable topsheet 3 can consist of a nonwoven material, e.g., a spunbond material of continuous filaments; a meltblown material, a bonded carded fibrous web or a perforated plastic film, The liquid impermeable backsheet 2 may consist of a plastic film, a nonwoven material coated with a liquid impervious material or a hydrophobic nonwoven material, which resists liquid penetration. The liquid impermeable backsheet 2 may also be a vapour permeable material.

The topsheet 3 and the backsheet material 2 have a somewhat greater extension in the plane than the absorbent body 4 and extend outside the edges thereof. The layers 2 and 3 are connected to each other wit the projecting portions thereof, e.g., by gluing or welding by heat or ultrasonic.

The absorbent body 4 can be of any conventional kind. Examples of commonly occurring absorbent materials are cellulose fluff pulp, tissue layers, highly absorbent polymers (so called superabsorbents), absorbent foam materials, absorbent nonwoven materials or the like. It is common to combine cellulose fluff pulp with superabsorbents in an absorbent body. It is also common to have absorbent bodies comprising layers of different material with different properties with respect to liquid acquisition capacity, liquid distribution capacity and storage capacity. It is well known to the person skilled in the art and does therefore not have to be described in detail. The thin absorbent bodies, which are common in for example baby diapers ad incontinence guards often, comprise a compressed mixed or layered structure of cellulose fluff pulp and superabsorbent.

The diaper/incontinence guard is intended to enclose the lower part of the wearer's trunk like a pair of absorbent pants. It comprises a front portion 5, intended during use to be worn on the frat part of the user's body, a roar portion 6, intended during use to be worn on the rear part of the user's body, and a crotch portion 7 located between the front and rear portions and which is intended to be worn in the crotch part of the user between the legs. The front portion 5 exhibits a pair of tape tabs 8 or another kind of fastening means such as hook and loop fasteners.

A pair of belt portions 9, 10 are with one end attached, e.g., glued or ultrasonically welded, to the rear portion 6 of the diaper. The belt portions 9, 10 are with their opposite ends intended to be fastened together by means of first fastening means 11, e.g. a hook-and-loop type fastener or tape tabs, intended to be attached against the outsides of the opposite belt portion. The second fastening means 8 of the front portion 5, such as for instance hook and loop fasteners or tape tabs, is intended to be attached against the outsides of the belt portions 9, 10 in order to fasten together the diaper/incontinence guard to the desired pantlike shape.

The width of the belt portions 9, 10 should be between 3-20 cm, preferably between 7-15 cm. The belt portions 9, 10 comprise according to an embodiment a laminate, wherein a carrier material forms the outside of the belt and a soft nonwoven forms the inside of the belt, intended to be in direct contact with the skin of the user. A suitable nonwoven material can be a spunbond material of e.g., polypropylene or polyethylene fibres. Conjugate fibres may also be used. Another suitable nonwoven material can be a carded thermobonded material of e.g., polypropylene, polyester or conjugate fibres. A plastic film or another suitable material e.g., nonwoven, may be used as carrier material. The carrier material should be adapted to function as a reception surface for both the attachment means 8 and 11, wherein in the case where these are tape tabs, aplastic film is suitable. In the case wherein other kinds of fastenings means is used instead of tape tabs, e.g., a hook and loop type fastener, another kind of carrier material is used, in particular a nonwoven material, which can act as a reception surface for the fastening means in question. Separate reception surfaces for fastening means could also be arranged on the outsides of the belt.

The belt portions 9, 10 are each provided with at least one indicium 12. The indicia 12 on the opposite belt portions are symmetrically placed in the longitudinal direction of the belt portions 9, 10) on a certain distance from the attachment 13 of each belt portion 9, 10 to the rear portion 6 of the diaper/incontinence guard. Thus, each indicium 12 on one belt potion 9 has its corresponding indicium on the opposite belt portion 10, placed on essentially the same distance from the attachment 13 of each belt portion 9, 10 to the rem portion 6 of the article. Upon application of the article the belt portions 9, 10 are first attached around the waist of the wearer and said indicia 12 on the different belt portions 12 are fitted against each other and are levelled against the navel on the user. Thereby is ensured that the centre line 14 of the article is located at the centre of the back of tie user, since the position of the navel gives the centre position on the front side of the user. Thus, the share of the belt portion 9 extending in one direction from the attachment 13 of the belt portion 9 to the rear portion 6 around the patient is equally large as the share of the belt portion 10 extending in the other direction around the patient.

For reasons of manufacturing, X indicia 12 are preferably located on the inside portion of the belt portion, but the indicia may be located on both sides or on the outsides of the belt portions. Preferably, there are two or more indicia 12 on each belt portion 9, 10, so that an adaptation of the circumference of the belt to the size of the user can be performed. Said indicia 12 can in pairs consist of stripes, lines, dots, symbols, letters, ornamental designs, images, etc. having the same or different colour. Said indicia 12 can consist of the same pattern but in pairs have different colours.

The invention is of course not limited to the above described and on the drawing showed embodiments, but can be modified within the scope of the claims.

1. Absorbent article such as a diaper and an incontinence guard comprising a liquid permeable topsheet (3), a liquid impermeable backsheet (2) ad an absorbent body (4) enclosed therebetween, said article having a front portion (5), a rear portion (6) and a crotch portion (7) therebetween, and other is provided with a pair of belt portions (9, 10) attached to the rear portion (6) of the article and which are intended to be fastened together around the waist of the wearer, wherein the one belt portion (10) carries first fastening means (11) intended to be attached against the opposite belt portion (9) and where said front portion (5) exhibits second fastening means (8) intended to be attached to the belt portions (9, 10), in such a way that the article will assume a pantlike shape, where the belt portions (9, 10) form a part of the waist portions of the pant,
characterised in,
that each belt portions (9, 10) are provided with at least one indicium (12), being placed on a appropriate distance from the attachment (13) of each belt portion (9, 10) to the rear portion (6) of the article, whereby each indicium (12) on one belt portion (9) has its corresponding indicium on the opposite belt portion (10).

2. Absorbent article according to claim 1,
characterised in,
that said indicia (12) are an ornamental design.

3. Absorbent article according to claim 1,
characterised in,
that said indicia (12) are symbols.

4. Absorbent article according to claim 1,
characterised in,
that said indicia (12) are coloured indicia.
